(19) United States
(12) Patent Application Publication
(10) Pub. No.: US 2025/0029675 A1
(43) Pub. Date: Jan. 23, 2025

(54) MEDICAL INFORMATION PROCESSING APPARATUS

(71) Applicant: Canon Medical Systems Corporation, Otawara-shi (JP)

(72) Inventors: Kohei WATANABE, Utsunomiya (JP); Koji HIRATA, Kasukabe (JP)

(73) Assignee: Canon Medical Systems Corporation, Otawara-shi (JP)

(21) Appl. No.: 18/775,720

(22) Filed: Jul. 17, 2024

(30) Foreign Application Priority Data
Jul. 21, 2023 (JP) 2023-119015

(51) Int. Cl. G16B 15/30; G16B 40/10; G16B 50/00; G16H 10/40

(52) U.S. Cl. CPC G16B 15/30; G16B 40/10; G16B 50/00; G16H 10/40

(57) ABSTRACT

According to one embodiment, a medical information processing apparatus includes processing circuitry, the processing circuitry configured to acquire first information relating to characteristics of an extracellular vesicle (EV) obtained from a subject to whom a drug has been administered, which has correlation with an origin tissue of the EV, acquire second information relating to presence or absence or content of the drug contained in the EV, and evaluate tissue migration of the drug by collating the first information and the second information with a database stored in advance.

### CROSS-REFERENCE TO RELATED APPLICATIONS

This application is based upon and claims the benefit of priority from Japanese Patent Application No. 2023-119015, filed Jul. 21, 2023, the entire contents of which are incorporated herein by reference.

### FIELD

Embodiments described herein relate generally to a medical information processing apparatus.

### BACKGROUND

Generally, the dosage of a drug is determined in terms of the body weight and the body surface area such that the drug concentration in the target tissue falls within the expected range based on pharmacokinetics. However, since the pharmacokinetics vary depending on various factors, the drug efficacy and the duration of action may vary among individuals even if the drug is taken at the predetermined dosage and administration. For example, it takes two to four weeks to confirm drug efficacy, with antidepressants and, if no effect cannot be recognized with the first drug, it may be necessary to switch the drug to a different drug and wait another two to four weeks. Alternatively, some drugs are administered as prodrugs and are metabolized in vivo to exert the efficacy. If the metabolic efficiency is lowered by impairment of the patient's liver function, etc., the medicinal ingredients may be lower than expected. As for anticancer drugs as well, the amount of drugs reaching cancer tissues is not constant depending on the nature of the cancer.

Currently, the blood concentration of a drug is commonly used as an indicator to determine the pharmacokinetics of drug. When the expected medicinal effects are not achieved, however, it is difficult to determine whether the drug has not migrated sufficiently to the target tissues or whether the action is lowered due to the patient's disease mechanism.

### BRIEF DESCRIPTION OF THE DRAWINGS

FIG. 1 is a block diagram showing an example of a first embodiment.

FIG. 2 is a block diagram showing an example of a second embodiment.

FIG. 3 is a block diagram showing an example of a third embodiment.

FIG. 4 is a block diagram showing an example of a fourth embodiment.

FIG. 5 is a workflow for an example of a fifth embodiment.

FIG. 6 is a schematic diagram showing an example of a capture mechanism of the fifth embodiment.

FIG. 7 is a block diagram showing an apparatus of Example 1.

FIG. 8 is a block diagram showing an apparatus of Example 2.

FIG. 9 is a block diagram showing an apparatus of Example 3.

FIG. 10 is a workflow showing an evaluation of tissue migration of a drug by an apparatus of Example 4.

FIG. 11 is a workflow showing an evaluation of tissue migration of a drug by an apparatus of Example 5.

### DETAILED DESCRIPTION

In general, according to one embodiment, a medical information processing apparatus comprises processing circuitry, the processing circuitry configured to: acquire first information relating to characteristics of an extracellular vesicle (EV) obtained from a subject to whom a drug has been administered, which has correlation with an origin tissue of the EV; acquire second information relating to presence or absence or content of the drug contained in the EV; and evaluate tissue migration of the drug by collating the first information and the second information with a database stored in advance.

Embodiments will be described hereinafter with reference to the accompanying drawings.

### First Embodiment

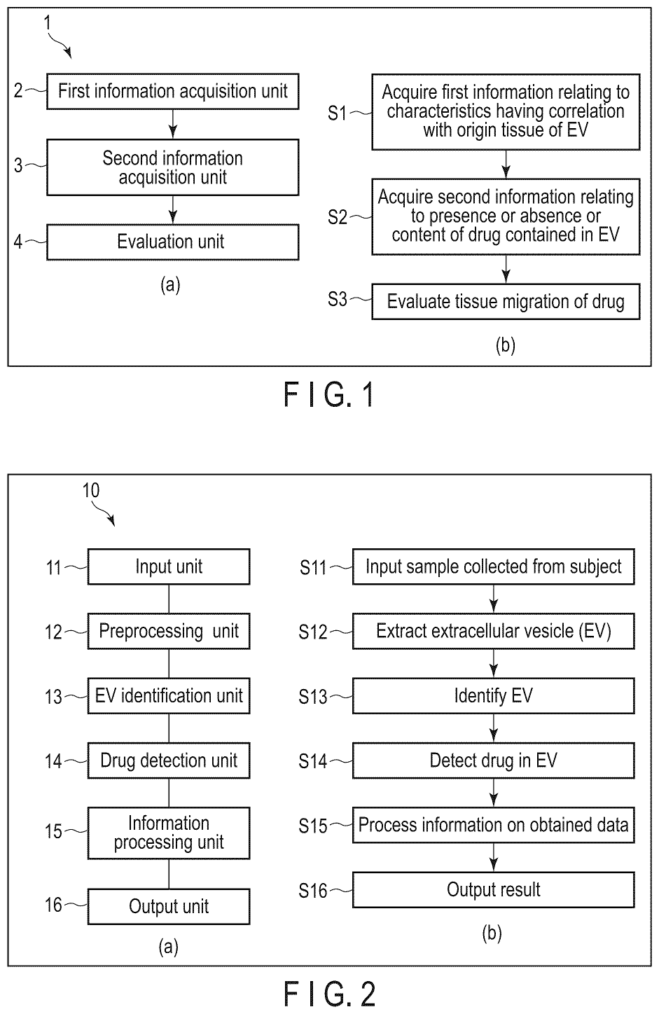

A medical information processing apparatus according to a first embodiment is an apparatus for evaluating the tissue migration of a drug. A medical information processing apparatus comprises processing circuitry. The processing circuitry is configured to acquire first information relating to characteristics of an extracellular vesicle (EV) obtained from a subject to whom a drug has been administered, which has correlation with an origin tissue of the EV, acquire second information relating to presence or absence or content of the drug contained in the EV, and evaluate tissue migration of the drug by collating the first information and the second information with a database stored in advance. One example of the medical information processing apparatus will be described with reference to FIGS. 1(a) and 1(b). A medical information processing apparatus 1 comprises a first information acquisition unit 2, a second information acquisition unit 3, and an evaluation unit 4. The first information acquisition unit 2, the second information acquisition unit 3 and an evaluation unit 4 are electrically connected to each other and compose processing circuitry. The first information acquisition unit 2 acquires first information (S1). The first information is information on characteristics of an extracellular vesicle (EV) obtained from a subject to whom the drug has been administered, which are correlated with an origin tissue of the EV. The second information acquisition unit 3 acquires second information (S2). The second information is information relating to the presence or absence or content of the drug contained in the EV. The evaluation unit 4 evaluates the tissue migration of the drug by collating the first information and the second information with a database stored in advance (S3). The database is, for example, stored in a memory included in the processing circuitry.

The first information acquisition unit 2 can be configured to be capable of acquiring characteristics having a correlation with the origin tissue of the EV. For example, the unit can be configured to acquire characteristics from the EV collected from the subject in advance. For example, the first information acquisition unit 2 can comprise an EV identification unit. The EV identification unit can be configured to obtain from the EV collected from the subject in advance, for example, information such as the size of individual EV, the amount of EV in the input sample, the relative amount of EV, and/or characteristics of the origin tissue thereof, and the like. In addition to the EV identification unit, the first information acquisition unit 2 may further comprise a configuration which is necessary to acquire the first information relating to characteristics having correlation with the origin tissue of EV or which assists in acquiring such first information. Alternatively, the first information acquisition unit 2 may be configured to acquire the first information by electrically receiving information relating to characteristics having correlation with the origin tissue of EV, which is acquired experimentally at any external detection apparatus.

The second information acquisition unit 3 is configured to acquire information relating to the presence or absence or content of a drug contained in the EV. For example, the second information acquisition unit 3 can be configured to acquire information relating to the drug, for the EV for which the first information has been acquired. Alternatively, the second information acquisition unit 3 can acquire the second information by receiving information relating to the presence or absence or content of the drug contained in the EV, which is acquired at any external detection apparatus as the second information.

The evaluation unit 4 can be electrically connected to the first information acquisition unit 2 and the second information acquisition unit 3, and can be configured to receive information from the first information acquisition unit 2 and the second information acquisition unit 3 and to evaluate the drug tissue migration, based on the received information. In the example in FIG. 1(a), concerning a connection of the first information acquisition part 2 and the second information acquisition unit 3 to the evaluation part 4, the first information acquisition part 2, the second information acquisition unit 3, and the evaluation unit 4 are connected in series in the order. However, the connection is not limited to this, and the first information acquisition unit 2 and the second information acquisition unit 3 may be connected to the evaluation unit 4 separately, i.e., in parallel. The evaluation unit 4 may further optionally comprise a display unit configured to output evaluation results and any information.

### Second Embodiment

A medical information processing apparatus according to the embodiment comprises an input unit introducing an extracellular vesicle (EV) obtained from a subject to whom a drug has been administered, an EV identification unit determining characteristics of the introduced EV, a drug detection unit measuring the presence or absence or content of the drug contained in the EV, and an information processing unit evaluating tissue migration of the drug by collating information obtained in the EV identification unit and the drug detection section with a database stored in advance.

One example of the medical information processing apparatus of a second embodiment will be described with reference to FIGS. 2(a) and 2(b). A medical information processing apparatus 10 includes an input unit 11, an optional preprocessing unit 12, an EV identification unit 13, a drug detection unit 14, an information processing unit 15, and an optional output unit 16 (FIG. 2(a)).

The input unit 11 comprises an aperture for physically inputting a sample obtained from the subject to whom the drug has been administered. The sample input to the input unit 11 includes an extracellular vesicle (EV) derived from the subject. For example, such a sample can be blood, plasma or serum, or an EV containing solution extracted by preprocessing. When it is necessary to preprocess the blood, plasma or serum in a state where signals related to the EV can be detected, the preprocessing unit 12 connected to the input unit 11 can be arranged. In this case, the preprocessing unit 12 is configured such that, for example, processing such as EV isolation, extraction, or washing is performed in a container. For example, the preprocessing unit 12 can comprise a liquid processing unit and a liquid storage tank in which an extract, a washing solution, and an EV maintenance fluid can be added and drained. When the medical information processing apparatus 10 does not comprise the preprocessing unit 12, the input unit 11 is connected to the EV identification unit 13. The preprocessing unit 12 receives a sample input from the input unit 11 (S11) and performs preprocessing, for example, extraction as desired (S12). The extracellular vesicle (EV) obtained in S12 is sent to the EV identification unit 13. S13 identifies the EV sent from S12 as desired (S13).

More specifically, the EV identification unit 13 receives the EV input from the input unit 11 or the EV obtained by the preprocessing unit 12, from the input unit 11 or the preprocessing unit 12. After that, the EV identification unit 13 obtains from the received EV the information relating to characteristics such as the size of individual EV, the amount of EV in the input sample, the relative amount of EV, and/or the origin tissue (S13), and the like. The extracellular vesicle (EV) is, for example, a microvesicle, an exosome, which is an extracellular vesicle derived from an endosome, and the like. They contain information from cell membranes and intracellular multivesicular bodies and are present in the blood of mammals such as humans. The EV includes information on origin organisms, such as adhesion or signaling molecules such as integrins, CD9, CD63, and CD81, immune-related molecules such as MHCI and MHCII, nucleic acids such as miRNA, mRNA, and DNA, enzymes, cytoskeletal proteins such as actin, tubulin, and cofilin, MVB components such as Alix, Tsg101, and clathrin, chaperones such as HSP70, HSP90, and HSP50. By obtaining these informations and the signals such as optical and electrochemical information of EV, the information on the origin tissue and the origin organ of the EV can be obtained.

For example, to estimate the origin tissue using surface antigens as biomarkers, for example, corresponding antibodies can be immobilized on a chip and detected by capturing origin tissue-specific antigens on the EV surface with the antibodies and/or by labeling the biomarkers on the EV surface with antibodies. Alternatively, mass spectrometry imaging may be used. In other words, the EV identification unit 13 may comprise, for example, a detection unit using an antigen-antibody reaction or a mass spectrometry imaging unit.

In addition, it has been reported that the EV is released from various tissues in living organisms and encapsulates nucleic acids and proteins retained by the origin tissues as well as taken substances. For example, it has been reported that the EV encapsulates and releases drugs taken up by cells. The drug detection unit 14 is an element that obtains information on such drugs possessed by the EV, i.e., information on the drugs taken up by the origin tissue of EV (S14). The drug detection unit 14 may be arranged in the medical information processing apparatus 10 as the same component as the EV identification unit 13 or may be arranged in the medical information processing apparatus 10 as a component different from the EV identification unit 13. Alternatively, the drug detection unit 14 and the EV identification unit 13 may be arranged in the medical information processing apparatus 10 as a single component. Therefore, the drug detection unit 14 may be connected to the EV identification unit 13, the preprocessing unit 12, and/or the input unit 11. The signals and/or information from the EV identification unit 13 and the signals and/or information from the drug detection unit 14 are sent to the information processing unit 15. The information processing unit 15 electrically communicates with the drug detection unit 14 and the EV identification unit 13. The information processing unit 15 can convert the signals and/or information obtained from the drug detection unit 14 and/or the EV identification unit 13 into information in a format defined in advance. For example, the information processing unit 15 comprises an arithmetic unit and a storage unit (not shown). The information processing unit 15 performs information processing such as calculation, based on information such as tables, arithmetic formulas, and associations between specific drugs and EV characteristics, which are stored in advance in the storage unit, furthermore according to programs stored in advance in the storage unit, as desired (S15). By the way, the above-described storage unit is also referred to as a database. For example, the database can store information groups, calculation formulas, and threshold values. The medical information processing apparatus 10 can output the results formed in the information processing unit 15 from an optional output unit 16. The output unit 16 may be a display unit, a sounding unit, a recording unit, a storage unit and/or a communication unit, and the like, to indicate the information obtained in the information processing unit 15 and the raw data collected in the EV identification unit 13 and/or the drug detection unit 14 for an operator or a preset target (S16). Examples of the output unit 16 can be a display, a speaker, a transmitter, a memory or the like. For example, the information processing unit 15 and the output unit 16 can both be CPU or computers.

A medical information processing apparatus evaluating the tissue migration of drugs with low invasiveness is provided by the medical information processing apparatus 10.

### Third Embodiment

A third embodiment will be described with reference to FIG. 3. A medical information processing apparatus 20 shown in FIG. 3 has basically the same configuration as the medical information processing apparatus 10. However, the medical information processing apparatus 20 includes a measurement unit 21 comprising an EV identification unit 13 and a drug detection unit 14, and a processing unit 22 comprising an information processing unit 15 and an output unit 16. The medical information processing apparatus 20 may have its operations controlled by a control unit (not shown) included in the processing unit 22 for each configuration and/or comprehensively.

A medical information processing apparatus evaluating the tissue migration of drugs with low invasiveness is provided by the medical information processing apparatus 20.

### Fourth Embodiment

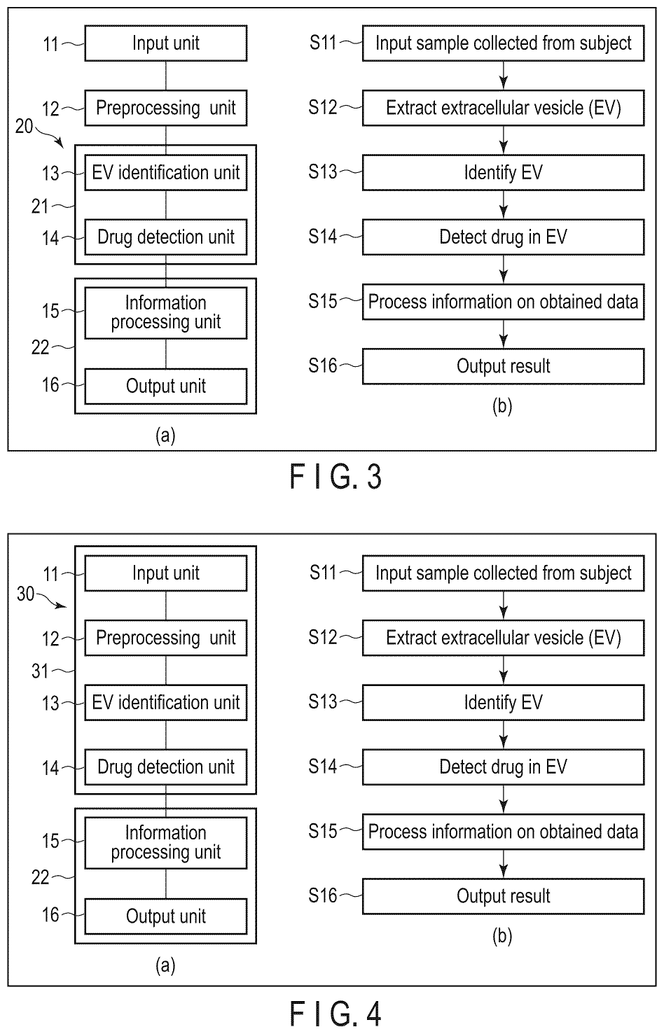

A fourth embodiment will be described with reference to FIG. 4. A medical information processing apparatus 30 shown in FIG. 4 has basically the same configuration as the medical information processing apparatuses 10 and 20. However, each component included in them is largely included in two units and provided in the medical information processing apparatus 30. In other words, the medical information processing apparatus 30 comprises a reaction unit 31 that performs a first procedure such as material reaction and liquid delivery, and a processing unit 22 that performs a second procedure such as electrical operation and control. The reaction unit 31 that performs the first procedure comprises an input unit 11, an optional preprocessing unit 12, an EV identification unit 13, and a drug detection unit 14. The processing unit 22 comprises an information processing unit 15 and an output unit 16. The medical information processing apparatus 30 may have its operations controlled by a control unit (not shown) included in the arithmetic unit 22 for each configuration and/or comprehensively.

A medical information processing apparatus evaluating tissue migration of drugs with low invasiveness is provided by the medical information processing apparatus 30.

### Fifth Embodiment

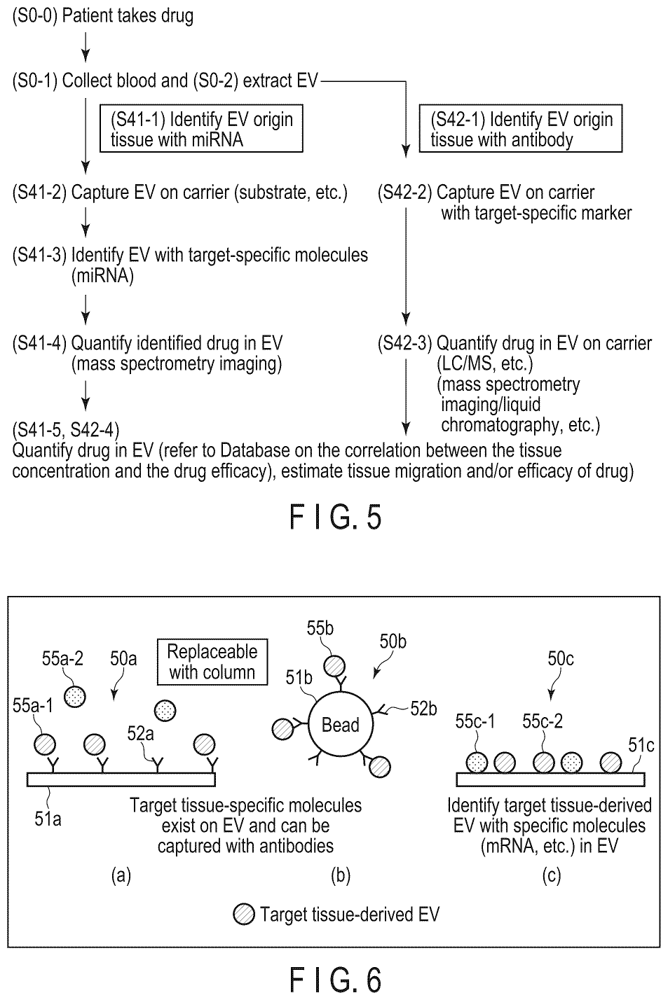

The fifth embodiment is a further example of a method of evaluating tissue migration of a drug using the medical information processing apparatus 10, 20, or 30. This method will be described with reference to FIG. 5. The medical information processing apparatus 10, 20, or 30 used in the fifth embodiment is an example of using a medical information processing apparatus A that does not include the preprocessing unit 12, which is optionally included in the above-described embodiments. A process prior to inputting a sample to the medical information processing apparatus A will be as follows. A patient takes a drug (S0-0). Blood is collected from the patient (S0-1). EV is extracted from the obtained blood (S0-2). (S0-0) and (S0-1) of the above procedure correspond to medical activities. (S0-0) and (S0-1) are not included in the procedure and the method of evaluating the tissue migration of drugs according to the fifth embodiment, except when they are used in a method including medical treatments or when there are special circumstances intended for medical treatments.

By the way, an example in which the process of extracting the EV from the blood collected from a patient is not included either is presented in the fifth embodiment. As described above, however, the process of extracting the EV from the obtained blood can also be included in the method according to the embodiment as desired. In such a case, the configuration of the medical information processing apparatus is the same as that described above.

In addition, in the fifth embodiment, miRNA information (S41-1) and/or antibody information (S42-1) to identify the origin tissue of EV is selected as the information obtained in the EV identification unit 13. To obtain such information, the EV identification unit 13 comprises a capture mechanism for capturing the EV. The EV obtained in advance is captured on a carrier (S41-2 and S42-2). An example of the capture mechanism is shown in FIG. 6. By the way, as described above, the EV identification unit 13 comprises a reaction field for conducting biochemical reactions therein. Such a reaction field is formed on a support body that can support liquid in a vessel, in a convex portion, in a channel, in a column, or on a flat plate.

As shown in FIG. 6(a), an EV capture mechanism 50a for obtaining antibody information comprises a plate-shaped support body 51a and antibodies 52a immobilized on a surface of the support body 51a. For example, antibodies of one type selected based on predicted pharmacokinetics of the drug to be evaluated may be immobilized on the support body 51a, or antibodies of a plurality of types may be immobilized to be identified based on any kinetics, on the support body 51a, or one or more antibodies selected at random may be identifiably immobilized on the support body 51a. In the reaction field, EV55a-1 is captured by the antibody 52a having corresponding anti-EV55a-1 characteristics. In contrast, EV55a-2, for which the corresponding antibody is not immobilized on the support body 51a, is not captured by the capture mechanism 50a but is ejected. The EV can also be captured selectively by this mechanism. Alternatively, a plurality of EV contained in the sample can be captured comprehensively depending on the type of antibody to be immobilized. The support body 51a may be a part of a wall surface of the column or channel or may be a part of the upper surface of the flat plate.

Alternatively, as shown in FIG. 6(b), the EV capture mechanism 50b for obtaining the antibody information may comprise a particle 51b such as a bead, and antibodies 52b immobilized on a surface of the particle 51b.

In contrast, as shown in FIG. 6(c), for example, the capture mechanism 50c can comprise any known nucleic acid capture means to obtain miRNA information. For example, a wall surface 51c that is in contact with the reaction field may be coated with a coating agent or modified with a specific substituent, or a complementary nucleic acid may be immobilized on the wall surface 51c. The information from the miRNA captured by the capture mechanism 50c can be obtained by using known target-specific molecules that identify miRNA.

For example, as described above, in S41-3, EV captured in S41-2 is identified with target-specific molecules (S41-3). For example, the information to be identified can be whether it is a miRNA, whether it is a specific type of miRNA, or whether it is a type of miRNA.

Then, as shown in FIG. 5, the drug contained in the EV identified by the EV identification unit 13 is quantified by the drug detection unit 14 (S41-4) (S42-3). The drug detection means that can be used commonly in methods utilizing both indicators of miRNA and antibodies is, for example, known mass spectrometry imaging. The drug is detected and/or measured while immobilized on the capture mechanisms 50a, b, and c.

In mass spectrometry imaging, the matrix, which is an ionizing agent, is applied and set in a mass spectrometry imaging apparatus, a laser beam is applied to a designated area, and the mass spectrum acquisition is repeated to obtain overall information. Each molecular ion peak is analyzed based on the obtained mass spectral information, and the distribution of compounds, which are targets, can be thereby obtained. The analysis targets are mainly animal tissues, human clinical specimens, three-dimensional model tissues (skin, tumors, etc.), plants (leaves, seeds, etc.), foods, etc., and visualization of low molecular weight metabolites, visualization of low molecular weight drugs, quantitative analysis, imaging of other special samples (e.g., hard tissues, microtissues, insects, etc.) and drugs, and the like can be performed.

When using antibodies, drugs may be quantified using liquid chromatography.

Then, in (S41-5) and (S42-4), the data obtained in the EV identification unit 13 and the drug detection unit 44 are sent to the information processing unit 15, which is electrically connected. The amount of drug in the EV is quantified or modified to relative values or standardized and then converted into comparable data in the information processing unit 15. This conversion is performed by using a database on the correlation between the tissue concentration and the drug efficacy, a table or formula that correlates the tissue migration with the drug efficacy, or other predetermined threshold values or formulas, and comparing or collating with them.

The results calculated by the information processing unit 15 are sent to the output unit 16 for display as desired (not shown in FIG. 5).

### Example 1

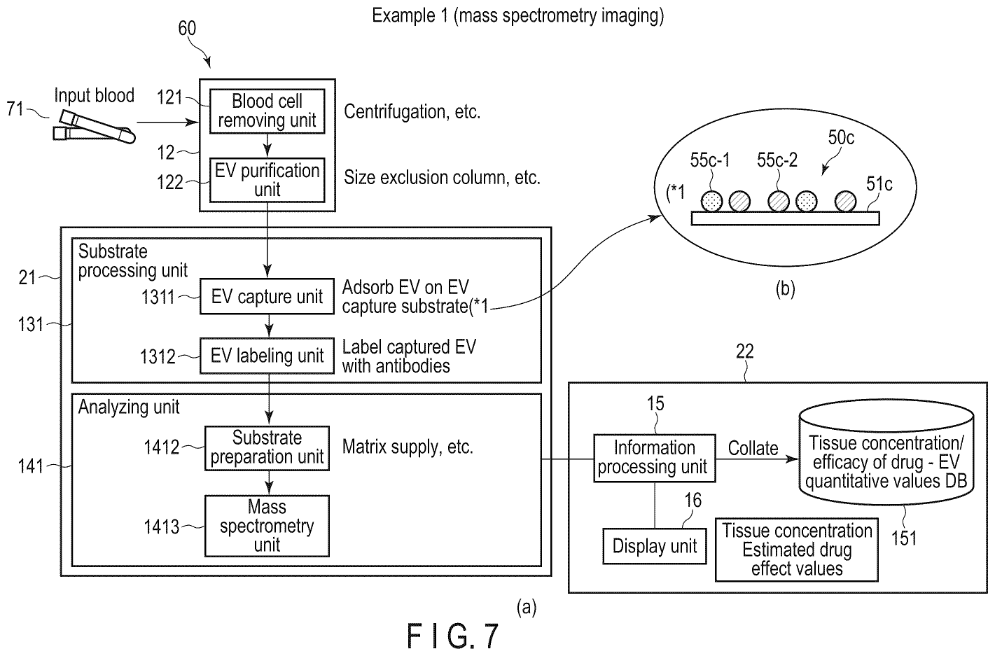

The medical information processing apparatus using mass spectrometry imaging of Example 1 will be described with reference to FIG. 7. As shown in FIG. 7(a), a medical information processing apparatus 60 of Example 1 comprises an input unit 11 (not shown), which is an opening for inputting input blood 71 as a sample, a preprocessing unit 12, a measurement unit 21, and a processing unit 22. The preprocessing unit 12 comprises a blood cell removing unit 121 and an EV purification unit 122. The blood cell removing unit 121 removes blood cells from the input blood 71. For example, the blood cell removing unit 121 may be a centrifugation mechanism, a membrane, or filter mechanism. The EV purification unit 122 may be, for example, a size exclusion column, an ion exchange column, or the like. The measurement unit 21 comprises a substrate processing unit 131 and an analyzing unit 141. The substrate processing unit 131 comprises an EV capture unit 1311 and an EV labeling unit 1312. The EV capture unit 1311 may be comprised of the same mechanism as the above-described EV capture unit. For example, FIG. 7(b) comprises a capture mechanism 50c comprising the same configuration as a capture mechanism 50c shown in FIG. 6(c). The EV labeling unit 1312 is configured to label the EV captured by the capture mechanism 50c with an antibody. The analysis unit 141 comprises a substrate preparation unit 1412 and a mass spectrometry unit 1413. The analysis unit 141 is configured such that Mass spectrometry be performed in the analytical unit 141. The matrix supply is performed and the mass of the drug is analyzed by a mass spectrometry imaging system.

The information obtained in the measurement unit 21 is sent to the processing unit 22. The measurement unit 21 and the processing unit 22 are electrically connected to each other. The processing unit 22 comprises an information processing unit 15, a storage unit 151, and a display unit 16, which are electrically connected to each other. Data obtained by the measurement unit 21 is sent to the information processing unit 15. The information processing unit 15 collates the data with a correspondence table between the tissue concentration and the drug efficacy, correspondence between actual measurement data and EV quantitative values which are stored in the storage unit 151, and the like. Alternatively, the information processing unit 15 processes the data received from the measurement unit 21 based on predetermined calculation formulas, and the like. The final results obtained in the information processing unit 15 are sent to the display unit 16 for display. Examples of the final results may be the tissue concentration of the drug and/or estimated drug efficacy values, and the like.

A medical information processing apparatus evaluating the tissue migration of drugs with low invasiveness is provided by the medical information processing apparatus 60.

### Example 2

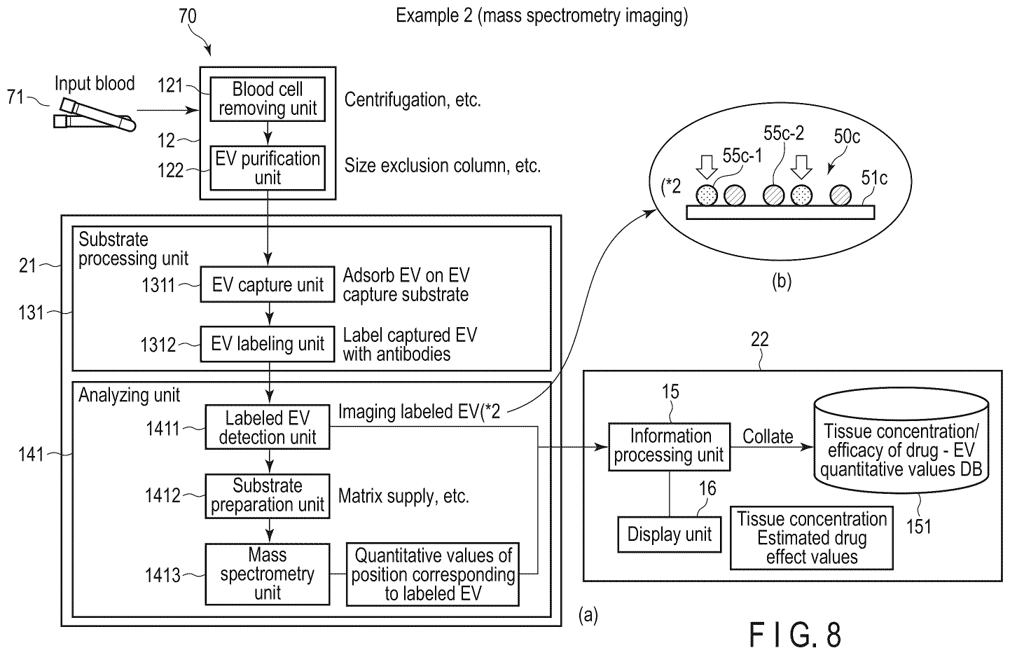

A medical information processing apparatus 70 using mass spectrometry imaging of Example 2 will be described with reference to FIG. 8. The medical information processing apparatus 70 has the same configuration as the medical information processing apparatus 60, except for further comprising a labeled EV detection unit 1411 in the measurement unit 21 of the medical information processing apparatus 60 of Example 1 shown in FIG. 7(a). The labeled EV detection unit 1411 communicates with the EV labeling unit 1312 and the substrate preparation unit 1412. The EV captured and labeled with antibodies by the EV labeling unit 1312 is imaged by the mass spectrometry imaging system. In this case, the mass spectrometry imaging system may comprise, for example, an imaging apparatus unit using an optical microscope for imaging a two-dimensional position of the sample and a CCD imaging apparatus or a CMOS imaging apparatus, a stage unit for moving the sample, a sample introduction unit, an ionization unit for ionizing the molecules in the sample, a mass separation unit for separating ionized molecules according to m/z, a detection unit for detecting the separated ions, an imaging mass spectrometry data storage unit, a data processing unit for performing analysis and processing, and the like. The substrate preparation unit 1412 supplies the matrix, and the like for the obtained image, and quantitative values corresponding to the position information corresponding to the labeled EV are obtained in the mass spectrometry unit 1413. The obtained measured data is sent to the information processing unit 15. The subsequent processing is performed in the same manner as Example 1.

A medical information processing apparatus evaluating the tissue migration of drugs with low invasiveness is provided by the medical information processing apparatus 70.

### Example 3

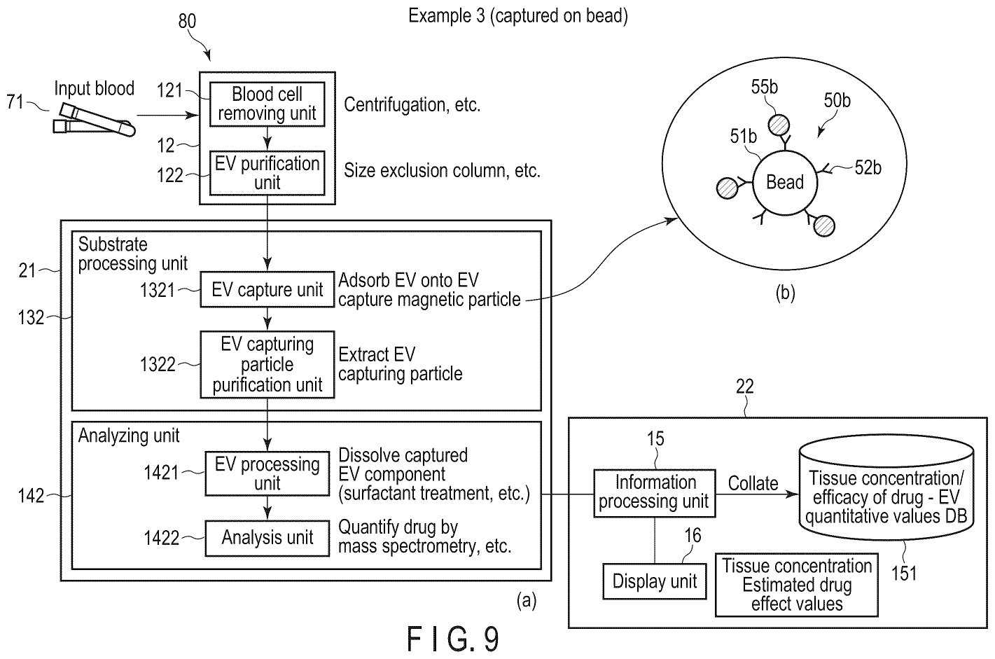

A medical information processing apparatus using a bead capturing mechanism of Example 3 will be described with reference to FIG. 9. As shown in FIG. 9(a), a medical information processing apparatus 80 of Example 1 comprises an input unit 11 (not shown), which is an opening for inputting input blood 71 as a sample, a preprocessing unit 12, a measurement unit 21, and a processing unit 22. The preprocessing unit 12 is the same as that described in Example 1.

The measurement unit 21 comprises a substrate processing unit 132 and an analyzing unit 142. The substrate processing unit 132 comprises an EV capture unit 1321 and an EV capturing particle purification unit 1322. The EV capture unit 1321 may be comprised of the same mechanism as the above-described EV capture unit. For example, FIG. 9(b) comprises a capture mechanism 50b using a bead capture mechanism comprising the same configuration as the capture mechanism (EV capturing particle) 50b shown in FIG. 6(b). In the present example, a magnetic particle is used as a bead for capturing the EV. In the EV capture unit 1321, the EV from the EV purification unit 122 is adsorbed onto the EV capturing magnetic particle. After that, in the EV capturing particle purification unit 1322, an EV capturing particle 50b is extracted utilizing the magnetism. The extracted EV capturing particles 50b is sent to the EV processing unit 1421. In the EV processing unit 1421, the captured EV component is dissolved by a surfactant treatment or the like and released from the capturing particle 50b. The released EV component is sent to the analysis unit 1422, where the drug is quantified by mass spectrometry or the like. The measurement results obtained in the analysis unit 1422 are sent to the information processing unit 15. The subsequent processing is performed in the same manner as Example 1.

A medical information processing apparatus evaluating the tissue migration of drugs with low invasiveness is provided by the medical information processing apparatus 80.

### Example 4

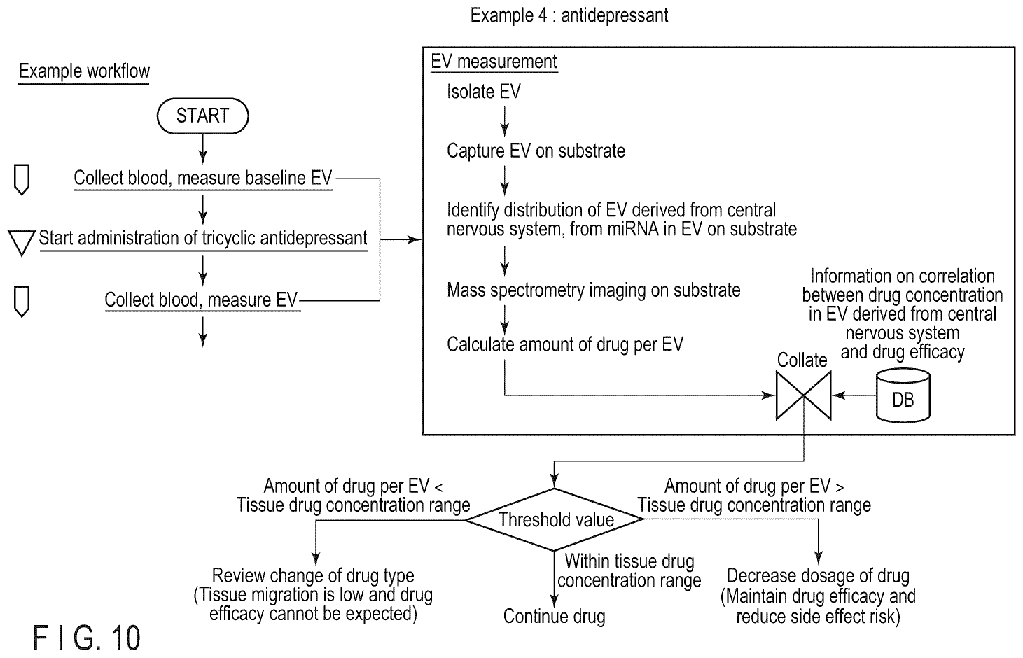

The tissue migration of an antidepressant will be evaluated using the medical information processing apparatus 60 of Example 1. As shown in FIG. 10, when the test is started, the drug content is first quantified for blood collected from a patient before administrating the antidepressant. This value is referred to as a baseline EV measurement value. The baseline EV measurement may be performed in the same manner as a quantification method performed on a blood sample to be described below.

After the baseline EV measurement value is obtained, administration of the antidepressant to the patient is started. Blood is periodically collected for monitoring. For example, a tricyclic antidepressant may be used as the antidepressant. The EV measurement value of the blood sample obtained by blood collection from the patient to which the drug has been administered is measured by the medical information processing apparatus 60 to evaluate the tissue migration of the drug.

The blood sample is input to the medical information processing apparatus 60, the EV is isolated in the preprocessing unit 12 and then captured on the substrate in the measurement unit 21, by the same procedure as that in Example 1 described above. Then, the distribution of the EV derived from the central nervous system is identified from the miRNA contained in the EV captured on the board. For this identification, the EV derived from the central nervous system may be identified from the EV group distributed using the type of miRNA as an indicator. The mass spectrometry imaging is performed for the identified EV, and the amount of drug per EV is calculated. The calculated amount of drug is collated with the information in the database stored in the storage unit 151, i.e., information on the correlation between the drug concentration and the drug efficacy in the EV derived from the central nervous system, in the processing unit 22. The specific determination is performed as follows. The amount of drug calculated by the measurement unit 21 is compared with the predetermined threshold value. If it is determined that “the amount of drug per EV<tissue drug concentration range”, “change in drug type needs to be considered” is displayed on the display unit 16 since “the tissue migration is low and the drug efficacy cannot be expected”. If it is determined that the amount of drug is “within the tissue drug range” as a result of comparison with the threshold value, “continuing the drug” is displayed on the display unit 16 since “the drug is appropriately migrated to the tissue and the drug efficacy can be expected”. If it is determined that “the amount of drug per EV>the tissue drug concentration range” as a result of comparison with the threshold value, “decreasing the dosage of drug” is displayed on the display unit 16 since “maintaining the drug efficacy and reducing the risk of side effects” is desired. The evaluation of the tissue migration of the antidepressant using the medical information processing apparatus 60 is ended by the display of the result on the display unit 16.

Thus, according to Example 4, the tissue migration of the antidepressant drug can be evaluated. The origin tissue can be examined by a biomarker (surface antigen markers/miRNA, etc.) held by the EV (extracellular vesicle). In addition, the EV may enclose and release the drug (e.g., paclitaxel) taken up by the cell when the EV is released from the cell. The evaluating of the tissue migration of drug with low invasiveness can be performed using a medical information processing apparatus, by using such a biological reaction.

### Example 5

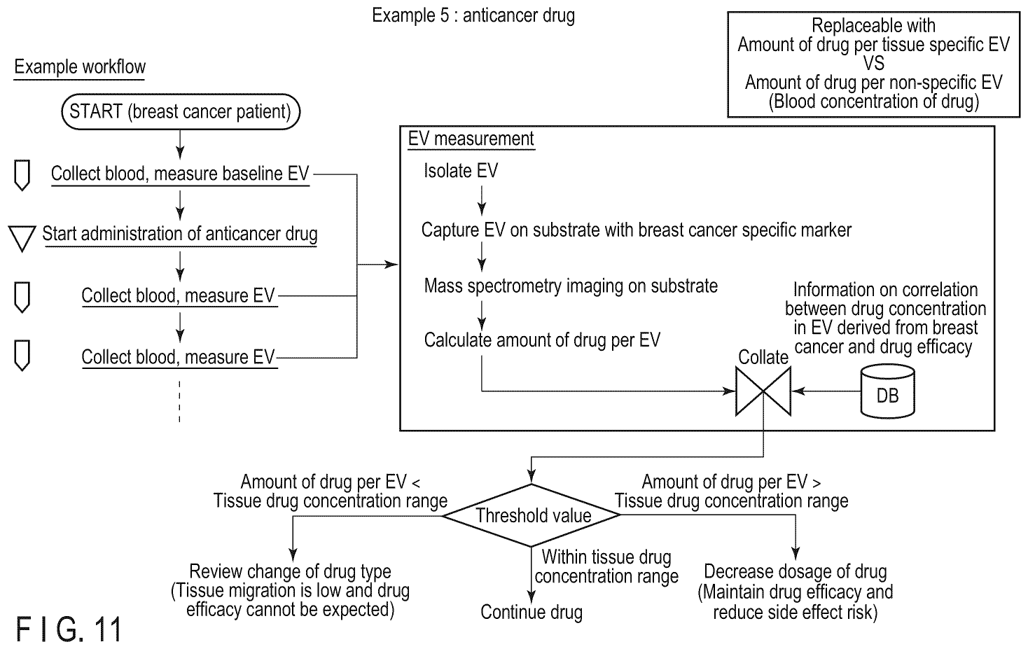

The tissue migration of the anticancer drug will be evaluated using the medical information processing apparatus 60 of Example 1. As shown in FIG. 11, when the test is started, the drug content is first quantified for blood collected from a breast cancer patient before administration of the anticancer drug. This value is referred to as a baseline EV measurement value. The baseline EV measurement may be performed in the same manner as a quantification method performed on a blood sample to be described below.

After the baseline EV measurement value is obtained, administration of the anticancer drug to the patient is started. Blood is periodically collected for monitoring. The EV measurement value of the blood sample obtained by blood collection from the patient to which the drug has been administered is measured by the medical information processing apparatus 60 to evaluate the tissue migration of the drug.

The blood sample is input to the medical information processing apparatus 60, and the EV is isolated in the preprocessing unit 12, by the same procedure as that in Example 1 described above. After that, the EV is captured on a substrate coated with a breast cancer specific marker in the measurement unit 21. Then, mass spectrometry imaging is performed for the EV captured on the substrate, and the amount of drug per EV is calculated. The calculated amount of drug is collated with the information in the database stored in the storage unit 151, i.e., the information on the correlation between the drug concentration in EV derived from breast cancer and the drug effects, in the processing unit 22. The specific determination is performed as follows.

The amount of drug calculated by the measurement unit 21 is compared with the predetermined threshold value. If it is determined that “the amount of drug per EV<tissue drug concentration range”, “change in drug type needs to be considered” is displayed on the display unit 16 since “the tissue migration is low and the drug efficacy cannot be expected”. If it is determined that the amount of drug is “within the tissue drug range” as a result of comparison with the threshold value, “continuing the drug” is displayed on the display unit 16 since “the drug is appropriately migrated to the tissue and the drug efficacy can be expected”. If it is determined that “the amount of drug per EV>the tissue drug concentration range” as a result of comparison with the threshold value, “decreasing the dosage of drug” is displayed on the display unit 16 since “maintaining the drug efficacy and reducing the risk of side effects” is desired. The evaluation of the tissue migration of the anticancer drug using the medical information processing apparatus 60 is ended by the display of the result on the display unit 16.

Thus, according to Example 5, the tissue migration of the anticancer drug can be evaluated. The origin tissue can be examined by a biomarker (surface antigen markers/miRNA, etc.) held by the EV (extracellular vesicle). In addition, the EV may enclose and release the drug taken up by the cell (e.g., anticancer drug) when the EV is released from the cell. The evaluating of the tissue migration of drug with low invasiveness can be performed using a medical information processing apparatus, by using such a biological reaction.

Since it is possible to evaluate the drug migration of the drug to the target tissue/off-target tissue by only collecting blood for the patient, the drug efficacy and side effects can be predicted with less burden. Therefore, the optimal drug administration method for each patient can be adjusted, and the improvement of the therapeutic efficacy, reduction in side effects, and the like can be expected.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising processing circuitry, the processing circuitry configured to:
acquire first information relating to characteristics of an extracellular vesicle (EV) obtained from a subject to whom a drug has been administered, which has correlation with an origin tissue of the EV;
acquire second information relating to presence or absence or content of the drug contained in the EV; and
evaluate tissue migration of the drug by collating the first information and the second information with a database stored in advance.

2. The medical information processing apparatus of claim 1, wherein the processing circuitry further configured to:
display a result obtained from the evaluation unit.

3. The medical information processing apparatus of claim 1, wherein
characteristics of the EV are information on the origin tissue.

4. The medical information processing apparatus of claim 1, wherein
characteristics of the EV are obtained from miRNA contained in the EV.

5. The medical information processing apparatus of claim 1, wherein
characteristics of the EV are obtained from an antigen presented on the EV.
